(19) United States
(12) Patent Application Publication
(10) Pub. No.: US 2023/0312472 A1
(43) Pub. Date: Oct. 5, 2023

(54) PROCESS FOR PREPARING REMIFENTANIL HYDROCHLORIDE

(71) Applicant: JOHNSON MATTHEY PUBLIC LIMITED COMPANY, London (GB)

(72) Inventors: Susan ANDREWS, Edinburgh (GB); Nicolas ARCHER, Edinburgh (GB)

(21) Appl. No.: 18/003,975

(22) PCT Filed: Jul. 2, 2021
(86) PCT No.: PCT/GB2021/051680

§ 371 Date: Dec. 30, 2022

(30) Foreign Application Priority Data
Jul. 2, 2020 (GB) 2010168.9

(51) Int. Cl. C07D 211/66

(52) U.S. Cl. CPC C07D 211/66

(57) ABSTRACT

The present invention provides a process for preparing remifentanil hydrochloride having an improved impurity profile.

The present invention provides a process for preparing remifentanil hydrochloride having an improved impurity profile.

EP383579B (to Glaxo Wellcome Inc.) describes the preparation of remifentanil base and hydrochloride.

The European Pharmacopoeia (edition 10.0) monograph for remifentanil hydrochloride (i.e. methyl 1-(3-methoxy-3-oxopropyl)-4-[phenyl(propanoyl)amino]piperidine-4-carboxylate hydrochloride) identifies a number of specified impurities. One of the specified impurities is remipropanamide i.e. methyl 4-[phenyl(propanoyl)amino]piperidine-4-carboxylate. Remipropanamide is listed as Impurity A.

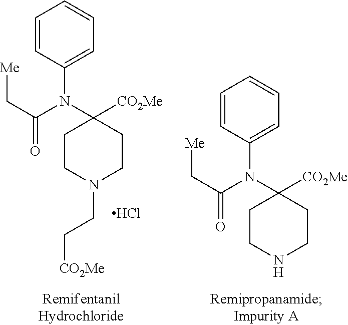 [SMILES: CCC(=O)N(c1ccccc1)C1(C(=O)OC)CCN(CCC(=O)OC)CC1 ; Cl ; CCC(=O)N(c1ccccc1)C1(C(=O)OC)CCNCC1]

The acceptance criterion for Impurity A in the monograph is not more than 0.2%. The acceptance criterion for unspecified impurities is not more than 0.10% for each impurity. The total for all impurities present in the remifentanil hydrochloride product cannot be more than 1.0%.

Remifentanil hydrochloride is a potent Active Pharmaceutical Ingredient (API). On plant, it is prepared under highly controlled conditions in a high potency suite and with a nurse on duty to observe those working in the suite so that action can be taken immediately in case of accidents. A low exposure limit has been set of 1 microgram/m³ Occupational Exposure Limit (OEL) over an eight-hour Time Weighted Average (TWA).

The inventors have developed an improved process which is more suited than prior art methods for the large-scale manufacture of remifentanil hydrochloride. The process of the invention is robust and produces remifentanil hydrochloride with consistent impurity profiles from batch to batch. It is desirable for the impurity profile of each batch to be well within the European Pharmacopoeia monograph limits so that the remifentanil hydrochloride exhibits a longer shelf-life, while reducing or eliminating the necessity for re-processing product which does not meet the required specification. Moreover, the process of the invention exhibits a greater tolerance to alcohol solvents which have been identified by the present inventors as producing unwanted impurities and, as such, preventing the production of remifentanil hydrochloride with predictable impurity profiles.

Definitions The term “ambient temperature” means one or more room temperatures between about 15° C. to about 30° C., such as about 15° C. to about 25° C.

The term “consisting” is closed and excludes additional, unrecited elements or process steps in the claimed invention.

The term “consisting essentially of” is semi-closed and occupies a middle ground between “consisting” and “comprising”. “Consisting essentially of” does not exclude additional, unrecited elements or process steps which do not materially affect the essential characteristic(s) of the claimed invention.

The term “comprising” is inclusive or open-ended and does not exclude additional, unrecited elements or process steps in the claimed invention. The term is synonymous with “including but not limited to”. The term “comprising” encompasses three alternatives, namely (i) “comprising”, (ii) “consisting”, and (iii) “consisting essentially of”.

The term “impurity” refers to a compound which is undesirably present and typically occurs in small quantities. The impurity may be present in the starting material, produced during the course of the reaction and/or is present in the product. Impurity A described above is an impurity named in the European Pharmacopeia for Remifentanil Hydrochloride.

The term “ketone solvent” refers to a liquid ketone in which remifentanil base is soluble but in which remifentanil hydrochloride is insoluble or substantially insoluble.

“Slurry” means a heterogeneous mixture of at least a portion of the solid remifentanil hydrochloride in one or more ketone solvents.

### DETAILED DESCRIPTION

The present invention provides a process for the preparation of remifentanil hydrochloride, the process comprising the step of combining a solution of remifentanil base in a ketone solvent with a solution of an alcohol solvent comprising hydrogen chloride, wherein one solution is added at a controlled rate to the other solution to form a reaction mixture comprising a precipitate of remifentanil hydrochloride.

Remifentanil base may be prepared using methods known in the art.

Suitable ketone solvents have boiling points at atmospheric pressure (i.e. 1.0135×10⁵ Pa) below about 160° C., such as below about 120° C. Examples include but are not limited to acetone, methyl ethyl ketone (MEK) also known as 2-butanone, methyl isobutyl ketone (MIBK) also known as 4-methyl-2-pentanone and diethylketone also known as 3-pentanone. In one embodiment, the ketone solvent is methyl isobutyl ketone.

Any suitable w/v ratio of remifentanil base to ketone solvent may be used provided a solution of remifentanil base in the ketone solvent is produced. The w/v ratio of remifentanil base to ketone solvent may be in the range of about 1 g of remifentanil base: about 1 to about 50 ml of ketone solvent, such as about 1 g of remifentanil base: about 5 to about 30 ml of ketone solvent, for example about 1 g of remifentanil base: about 7 to about 20 ml of ketone solvent. In one embodiment, the w/v ratio of remifentanil base to ketone solvent may be about 1 g remifentanil base: about 10 ml of ketone solvent.

The dissolution of remifentanil base may be encouraged through the use of an aid such as stirring or shaking. Additional solvent may be added to aid the dissolution of remifentanil base.

Suitable alcohol solvents have boiling points at atmospheric pressure (i.e. 1.0135×10⁵ Pa) below about 120° C., such as below about 100° C. and are capable of dissolving hydrogen chloride. In one embodiment, the alcohol solvent is anhydrous. Examples include but are limited to methanol, ethanol, n-propanol, isopropanol, butanol (n-, i- or t-), pentanols, cyclopentanol, hexanols, cyclohexanol or combinations thereof. Remifentanil hydrochloride is not very soluble in the ketone solvent. Without wishing to be bound by theory, it is believed that when one solution is added at a controlled rate to the other, reduced quantities of impurities form as the use of an alcohol solvent increases the solubility of remifentanil hydrochloride in the ketone/alcohol solvent reaction mixture which, as a result, both delays and better controls the crystallisation of remifentanil hydrochloride.

In one embodiment, the alcohol solvent is methanol. Methanol exhibits an additional advantage in comparison to other alcohol solvents in that a solution of hydrogen chloride in methanol produces a low quantity of hydrogen chloride vapour. Moreover and without wishing to be bound by theory, methanol appears to provide a stabilising effect during the downstream processing of remifentanil hydrochloride. This stabilising effect is explained in greater detail below.

Hydrogen chloride is a gas at room temperature. A solution of an alcohol solvent comprising hydrogen chloride may be prepared by dissolving hydrogen chloride in the alcohol solvent. This may be achieved by bubbling the hydrogen chloride through the alcohol solvent. Bubbling hydrogen chloride gas into an alcohol solvent is safer on plant than the direct gassing of remifentanil with hydrogen chloride as it is easier to control a solution than a gas and the risk of potent contaminated material being sucked back through gassing pipes is avoided.

The use of aqueous hydrochloric acid is not according to the invention. While the process of the invention has some tolerance to water, the inclusion of water in the process of the invention water is believed to cause the hydrolysis of the remifentanil molecule to form the impurity remifentanil acid (identified in the European Pharmacopoeia as Impurity C).

The hydrogen chloride may be bubbled into the alcohol solvent such that the solution is maintained at ambient temperature or less using cooling means such as an ice bath. In one embodiment, the solution may be maintained at one or more temperatures in the range of ≥ about 0° C. to about ≤30° C. In some embodiments, the solution may be maintained at one or more temperatures ≥ about 5° C. In some embodiments, the solution may be maintained at one or more temperatures ≥ about 10° C. In some embodiments, the solution may be maintained at one or more temperatures ≥ about 15° C. In some embodiments, the solution may be maintained at one or more temperatures ≤ about 30° C. In some embodiments, the solution may be maintained at one or more temperatures ≤ about 25° C. In some embodiments, the solution may be maintained at one or more temperatures ≤ about 20° C. In one embodiment, the solution may be maintained at one or more temperatures in the range of ≥ about 15° C. to ≤ about 30° C.

It is desirable to prepare a high concentration of hydrogen chloride in the minimum volume of alcohol solvent as this limits the volume of alcohol solvent added to the reaction mixture preventing possible adverse effects on the yield of remifentanil hydrochloride due to the solubility of remifentanil hydrochloride in the alcohol solvent. When the alcohol solvent is methanol, however, it is desirable that a compromise on the concentration of hydrogen chloride in the solvent is met as HCl and methanol can react to form water and methyl chloride over time. The higher the concentration, therefore, the more water and methyl chloride may be produced. The presence of methyl chloride is disadvantageous as it is a known impurity. While the process of the invention has some tolerance to water, the presence of water is usually disadvantageous because water is believed to cause the hydrolysis of the remifentanil molecule to form the impurity remifentanil acid (identified in the European Pharmacopoeia as Impurity C). In this respect, a water content of up to about 1.6% in the HCl/methanol solution has been demonstrated by the inventors as not being significantly detrimental to the process of the invention. In certain embodiments, however, it is desirable to limit the quantity of water to not more than about 0.5% in the HCl/methanol solution. The quantity of water present in the HCl/methanol solution can be determined by Karl Fisher titration. Moreover, as methyl chloride forms at the same rate as water, the methyl chloride content in the HCl/methanol solution can be deduced once the quantity of water has been calculated. The formation of water (and therefore methyl chloride) can be suppressed by storing the HCl/methanol solution in a freezer at a temperature of at least about −10° C., such as −18° C., before use in the process of the present invention. The inventors have found that HCl/methanol solution stored in a freezer at −18° C. for at least three months does not adversely impact the quality of remifentanil hydrochloride made.

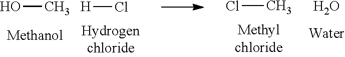

The concentration of hydrogen chloride in the alcohol solvent may be in the range of about 0.5 to about 5 molar, such as about 1 to about 4 molar, for example about 2 to about 3 molar.

In one embodiment, the hydrogen chloride is present in the reaction mixture in stoichiometric or in slight excess to the remifentanil base. When the hydrogen chloride is present in excess, it is calculated to provide a molar excess of at least 1% over the amount required for the stoichiometric reaction.

One solution is added at a controlled rate to the other solution. The controlled rate of addition does not include bulk addition in which one solution (for example, the acidic alcohol solution) is added to the other solution (for example, the solution of remifentanil base in a ketone solvent) in a single portion. The rate of addition may be any suitable rate capable of precipitating remifentanil hydrochloride but one which minimises or eliminates the entrapment of excess hydrogen chloride and/or remifentanil base in the remifentanil hydrochloride precipitate. The rate of addition may be adapted as appropriate by various parameters including the quantity and concentration of the acidic alcohol solution to be added, the scale of the reaction, the size of the reaction vessels, and the length of processing time.

In one embodiment, the solution of remifentanil base in a ketone solvent may be added at a controlled rate to the solution of an alcohol solvent comprising hydrogen chloride.

Alternatively, the solution of an alcohol solvent comprising hydrogen chloride may be added at a controlled rate to the solution of remifentanil base in a ketone solvent. This embodiment is typically more desirable with regards to health and safety as the handling of potent remifentanil base can be minimised.

Without wishing to be bound by theory, it is believed that if remifentanil hydrochloride precipitates out of solution suddenly (which may occur for example when a ketone solvent is solely used), excess hydrogen chloride may be trapped in the precipitated product. The excess hydrogen chloride may then be undesirably released on subsequent processing of the product and, for example, may catalyse the formation of at least one other impurity during that processing stage (for example, the remifentanil isopropyl ester impurity).

Data extracted from Example 6 of the present invention demonstrates that the pH of remifentanil hydrochloride samples obtained by adding the HCl/methanol solution in one charge are approximately 1 pH unit lower than those obtained when the HCl/methanol is added at a controlled rate. This data supports the theory that excess hydrogen chloride is undesirably trapped in the precipitated product during bulk/dump charge addition.

| | Dump charge addition | | |
|---|---|---|---|
| | Example 6 | Example 6 | Example 6 |
| | Sample A2* | Sample A3* | Sample A4* |
| pH of remifentanil | 4.22 | 4.31 | 4.21 |
| hydrochloride | | | |
| | Controlled rate of addition | | |
| | Example 6 | Example 6 | Example 6 |
| | Sample B2^{#} | Sample B3^{#} | Sample B4^{#} |
| pH of remifentanil | 5.55 | 5.51 | 5.62 |
| hydrochloride | | | |

*not according to the invention
^{#}according to the invention

On the other hand, if insufficient hydrogen chloride is added to the reaction mixture, remifentanil base may become trapped in the precipitated remifentanil hydrochloride product and may then (upon heating) undergo a reverse “Michael addition” to generate remipropanamide (Impurity A).

The controlled addition may take from about 5 minutes to about 60 minutes, such as about 7 to about 45 minutes, for example about 10 to about 30 minutes.

In one embodiment, the controlled addition is at a substantially constant rate. The rate of addition may depend on the scale of the reaction. For example, the rate of addition may be slower (i.e. take longer) on a smaller scale reaction compared to a larger scale reaction. In one embodiment, the rate of addition may be about 0.01 ml/minute to about 10 ml/minute, such as about 0.1 ml/minute to about 5 ml/minute. In another embodiment, the rate of addition may be about 10 ml/minute to about 100 ml/minute, such as about 20 ml/minute to about 80 ml/minute, for example about 30 ml/minute to about 70 ml/minute.

When the solution of an alcohol solvent comprising hydrogen chloride is added to the solution of remifentanil base in a ketone solvent, the pH of the reaction mixture will decrease from about pH 8 to an acidic pH. The inventors have found that the reaction is typically complete when the remifentanil hydrochloride reaction mixture has a pH of about 1.5 to about 2. It has been determined that about 1 mole equivalent of hydrogen chloride has been added when the reaction mixture achieves a pH in this range. In order to accurately target a pH in this range, the majority (e.g. about 70%, about 80% or about 90%) of the initial hydrogen chloride containing alcohol solution may be added to the reaction mixture as described above, the pH of the reaction mixture checked, the remaining portion (e.g. about 30%, about 20%, or about 10%) added to the reaction mixture as described above, followed by a final check of the pH. If the pH is less than about 1.5, excess hydrogen chloride has typically been added to the reaction mixture. If the pH is greater than about 2, insufficient hydrogen chloride has typically been added and the reaction mixture requires more hydrogen chloride containing alcohol solution to be added. Water wet narrow range pH papers (e.g. about 1.7 to about 3.8) may be used.

During the addition of one solution to the other, the temperature of the reaction mixture may be maintained at ambient temperature or less. In one embodiment, the reaction mixture may be maintained at one or more temperatures in the range of ≥ about 0° C. to about ≤30° C. In some embodiments, the reaction mixture may be maintained at one or more temperatures ≥ about 1° C. In some embodiments, the reaction mixture may be maintained at one or more temperatures ≥ about 2° C. In some embodiments, the reaction mixture may be maintained at one or more temperatures ≥ about 3° C. In some embodiments, the reaction mixture may be maintained at one or more temperatures ≥ about 4° C. In some embodiments, the reaction mixture may be maintained at one or more temperatures ≥ about 5° C. In some embodiments, the reaction mixture may be maintained at one or more temperatures ≤ about 30° C. In some embodiments, the reaction mixture may be maintained at one or more temperatures ≤ about 25° C. In some embodiments, the reaction mixture may be maintained at one or more temperatures ≤ about 20° C. In some embodiments, the reaction mixture may be maintained at one or more temperatures ≤ about 15° C. In one embodiment, the reaction mixture may be maintained at one or more temperatures in the range of ≥ about 15° C. to ≤ about 30° C.

The reaction mixture may be optionally stirred during the controlled addition. Stirring may be continued for a further period of time after the addition is complete e.g. for about 1 minute to about 1 hour, such as about 5 minutes.

The process may be carried out under an inert atmosphere (for example, nitrogen or argon).

On completion of the process, the remifentanil hydrochloride may be recovered by filtering, decanting or centrifuging. Howsoever the product of the invention is recovered, the separated product may be washed with solvent (e.g. one or more of the ketone solvents described above, such as MIBK) and dried. Drying may be performed using known methods, for example, at temperatures in the range of about 10° C. to about 60° C., such as about 20° C. to about 40° C., for example, ambient temperature optionally under vacuum (for example about 1 mbar to about 30 mbar) for about 1 hour to about 72 hours. Alternatively, the product may be dried by pulling nitrogen through e.g. a filter cake of the product for about 1 hour to about 72 hours. It is preferred that the drying conditions are maintained below the point at which remifentanil hydrochloride degrades and so when remifentanil hydrochloride is known to degrade within the temperature, pressure ranges or conditions given above, the drying conditions should be maintained below the degradation temperature, vacuum or conditions.

The process may further comprise the step of treating the precipitate of remifentanil hydrochloride with a ketone solvent. The ketone solvent may be as described above. In one embodiment, the ketone solvent is methyl isobutyl ketone.

The inventors have found that treating remifentanil hydrochloride with a ketone solvent washes out excess hydrogen chloride which may be present in the remifentanil hydrochloride precipitate.

Any suitable w/v ratio of remifentanil hydrochloride to ketone solvent may be used. The w/v ratio of remifentanil hydrochloride to ketone solvent may be in the range of about 1 g of remifentanil hydrochloride: about 1 to about 50 ml of ketone solvent, such as about 1 g of remifentanil hydrochloride: about 5 to about 30 ml of ketone solvent, for example about 1 g of remifentanil hydrochloride: about 7 to about 20 ml of ketone solvent. In one embodiment, the w/v ratio of remifentanil hydrochloride to ketone solvent may be about 1 g remifentanil base: about 10 to about 15 ml of ketone solvent.

In one embodiment, the remifentanil hydrochloride may be slurried in a ketone solvent, such as methyl isobutyl ketone.

The treatment of remifentanil hydrochloride with a ketone solvent may be carried out at ambient temperature or less as described above.

The treatment step is carried out for a period of time until it is determined that no or substantially no excess hydrogen chloride remains. Completion of the treatment step may be determined by in-process analysis e.g. by checking the pH of each ketone solvent wash with water wet narrow range pH papers as described above. The remifentanil hydrochloride precipitate may be treated with the ketone solvent one or more times (e.g. 2, 3, 4, 5 or more times). Typically, the treatment step is complete within about 2 hours.

The treatment step may optionally comprise stirring e.g. for about 1 minute to about 1 hour.

On completion of the process, the remifentanil hydrochloride may be recovered, washed and dried as described above.

The process may further comprise recrystallising the precipitate of remifentanil hydrochloride from one or more alcohol solvents. The alcohol solvent may be as described above. In one embodiment, the alcohol solvent may be a propyl alcohol, such as n- or isopropyl alcohol.

Any suitable w/v ratio of remifentanil hydrochloride to alcohol solvent may be used. The w/v ratio of remifentanil hydrochloride to alcohol solvent may be in the range of about 1 g of remifentanil hydrochloride: about 1 to about 50 ml of alcohol solvent, such as about 1 g of remifentanil hydrochloride: about 5 to about 30 ml of alcohol solvent, for example about 1 g of remifentanil hydrochloride: about 7 to about 25 ml of alcohol solvent. In one embodiment, the w/v ratio of remifentanil hydrochloride to alcohol solvent may be about 1 g remifentanil base: about 10 to about 20 ml of alcohol solvent.

The recrystallisation may comprise dissolving the precipitate of remifentanil hydrochloride in one or more alcohol solvents to form a solution (for example, by heating the reaction mixture to reflux) and allowing the remifentanil hydrochloride to precipitate out of solution (e.g. by allowing the reaction mixture to cool to ambient temperature). The purified remifentanil hydrochloride product may be recovered, washed (e.g. with one or more alcohol solvents described above) and dried as described above.

Without wishing to be bound by theory, if the remifentanil hydrochloride solid undesirably contains excess hydrochloride trapped within it, the hydrochloride may be released during the recrystallisation and catalyse the transesterification of remifentanil hydrochloride with the alcohol solvent. The alcohol solvent may be the solvent from which the remifentanil hydrochloride is recrystallised.

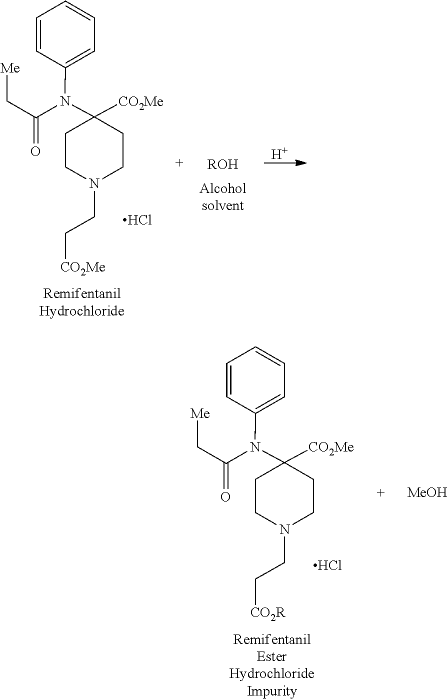

For example, when the remifentanil hydrochloride precipitate is recrystallised from isopropyl alcohol, the transesterification of remifentanil hydrochloride may produce remifentanil isopropyl ester hydrochloride as an impurity.

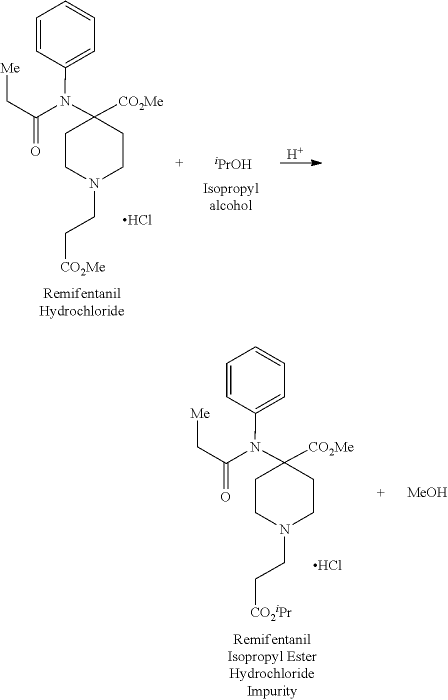

Alternatively or in addition, the alcohol solvent may originate from an earlier processing step which can be carried through to the recrystallisation step. For example, ethanol may be present in methyl isobutyl ketone (MIBK) as a residual impurity and may be introduced into the reaction mixture as a consequence of using MIBK in the formation of remifentanil hydrochloride. The ethanol therefore may react in the presence of HCl and undergo a transesterification reaction to form the remifentanil ethyl ester impurity.

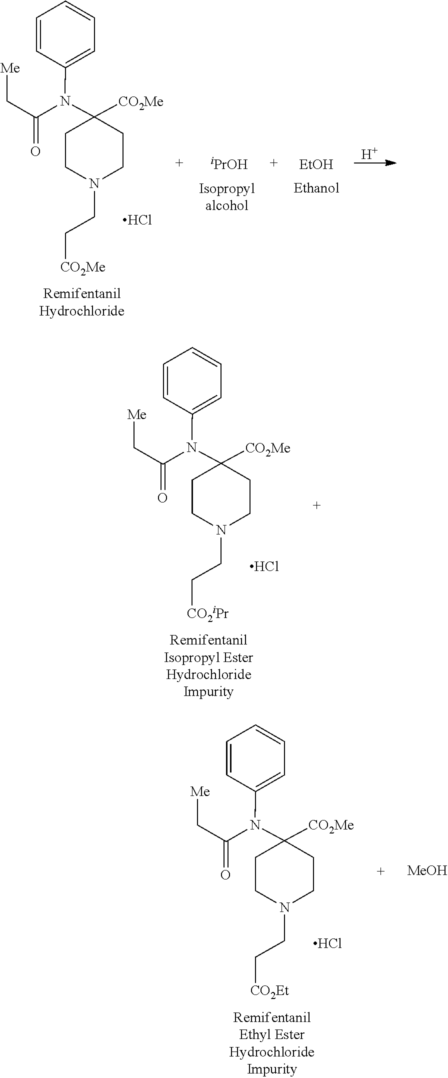

Typically, the recrystallisation is completed as quickly as possible. If the reaction mixture is heated to reflux, it is typically not desirable to hold the reaction mixture at reflux for an extended period of time (e.g. 30 minutes or more) as the remifentanil has a greater likelihood of undergoing the transesterification reaction and also some remifentanil may degrade to form the remipropanamide impurity.

When remifentanil hydrochloride is prepared using methanol comprising hydrogen chloride, the remifentanil hydrochloride precipitate produced may comprise residual methanol. Without wishing to be bound by theory, it is believed that the residual methanol provides a stabilising effect when the remifentanil hydrochloride is recrystallised from an alcohol solvent other than methanol itself. This is because the transesterification of remifentanil hydrochloride in the presence of residual methanol does not produce an impurity but rather reforms remifentanil hydrochloride itself.

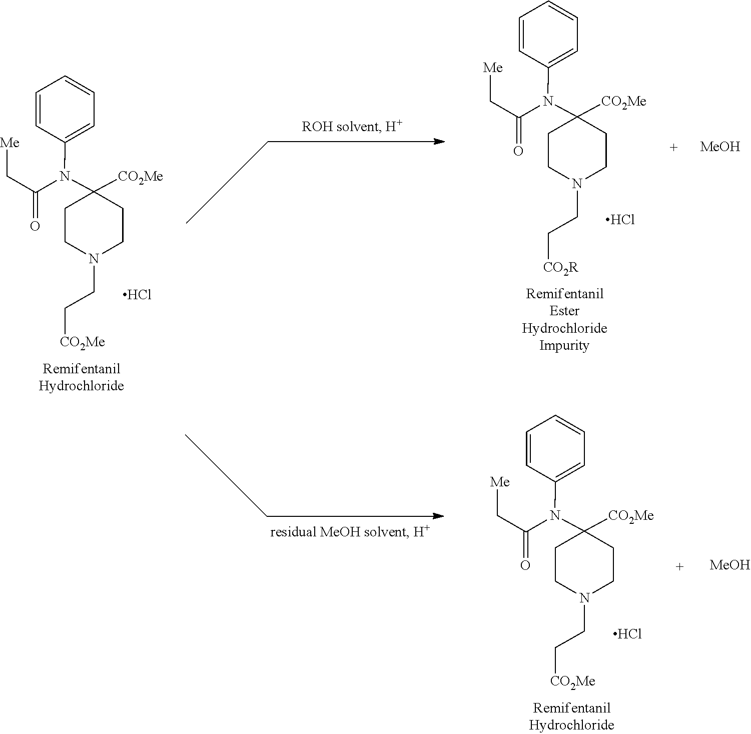

On completion of the process, the recrystallised remifentanil hydrochloride may be recovered, washed and dried as described above.

In carrying out the process of the invention, it is possible to obtain a product (remifentanil hydrochloride) with an improved impurity profile and, in certain embodiments, the recrystallisation step may be optional.

In one embodiment, it is possible to significantly reduce the levels of Impurity A (remipropanamide) in remifentanil hydrochloride, an impurity which must be controlled to particular levels specified in Official Monographs such as the European Pharmacopeia. For example, the European Pharmacopeia Monograph for Remifentanil Hydrochloride details that the acceptance criterion for Impurity A cannot be more than 0.2%. It is important to recognise, however, that the Official Monograph relates to remifentanil hydrochloride which is suitable for formulation and subsequent administration to a person. In this respect, the remifentanil hydrochloride ultimately prepared in a production campaign may have undergone several (or, indeed, many) purification treatments in order to reduce the level of Impurity A, as well as other impurities, to sufficiently acceptable low levels in order to conform to the required standard. The purification treatments therefore can typically result in extended processing times on plant and loss in product yield. In carrying out the process of the present invention, however, the formation of Impurity A can be minimised, thus reducing the requirement for further purification.

Example 4 (not according to the invention) shows that the direct gassing of a solution of remifentanil base with hydrogen chloride is not a process which produces consistent results. In this respect, variable quantities of remipropanamide are produced from batch to batch (see Samples A-G below). While the quantities of remipropanamide can be reduced by recrystallising the remifentanil hydrochloride Technical to form remifentanil hydrochloride Pure (see Sample H-J), the quantities of remipropanamide remaining are still not consistent from batch to batch. Additional processing e.g. by recrystallising remifentanil hydrochloride pure may reduce the remipropanamide level further (see Sample K) but this results in extended processing times on plant and loss in product yield as described above.

| | | RemiPPH by area % |
|---|---|---|
| Sample | Stage | 24-hour stress test |
| A | Remifentanil Hydrochloride Technical | 1.18 |
| B | Remifentanil Hydrochloride Technical | 2.90 |
| C | Remifentanil Hydrochloride Technical | 2.67 |
| D | Remifentanil Hydrochloride Technical | 0.89 |
| E | Remifentanil Hydrochloride Technical | 1.31 |
| F | Remifentanil Hydrochloride Technical | 1.18 |
| G | Remifentanil Hydrochloride Technical | 0.04 |
| H | Remifentanil Hydrochloride Pure | 0.19 |
| I | Remifentanil Hydrochloride Pure | 0.55 |
| J | Remifentanil Hydrochloride Pure | 0.30 |
| K | Recrystallised Remifentanil Hydrochloride | 0.09 |
| | Pure | |

Examples 5 and 6, however, demonstrate that consistently low quantities of remipropanamide are produced in the process according to the invention, even when insufficient hydrogen chloride (Example 5, Sample A10) or excess hydrogen chloride (Example 5, Sample B10) has been added:

| | 24-hour stress test | | | | |
|---|---|---|---|---|---|
| | Insufficient | Excess | | | |
| | HCl in | HCl in | | | |
| | HCl/methanol | HCl/methanol | Controlled rate of addition | | |
| | Example 5 | Example 5 | Example 6 | Example 6 | Example 6 |
| | Sample | Sample | Sample | Sample | Sample |
| | A10^{#} | B10^{#} | B2-1^{#} | B3-1^{#} | B4-1^{#} |
| Quantity of | 0.33 | 0.25 | 0.02 | 0.02 | 0.02 |
| remipropanamide | | | | | |
| impurity, area % | | | | | |
| by HPLC | | | | | |
| Total quantity of | 0.47 | 0.40 | 0.99 | 1.05 | 1.04 |
| impurities | | | | | |
| formed, | | | | | |
| % by HPLC | | | | | |

^{#}according to the invention

Example 6 evidences a reduction in the formation of the remifentanil isopropyl ester hydrochloride impurity in a process according to the present invention after subjecting samples of remifentanil hydrochloride to a 24-hour stress test. Example 6 also evidences a reduction in the total quantities of impurities formed after a controlled rate of addition of HCl/methanol (according to the invention) in contrast to a bulk addition of HCl/methanol (not according to the invention).

| | 24-hour stress test | | | |
|---|---|---|---|---|
| | Bulk addition | Controlled rate of addition | | |
| | Example 6 | Example 6 | Example 6 | Example 6 |
| | Sample | Sample | Sample | Sample |
| | A4-1* | B2-1^{#} | B3-1^{#} | B4-1^{#} |
| Quantity of IPA | 0.94 | 0.65 | 0.69 | 0.68 |
| ester impurity, | | | | |
| area % by HPLC | | | | |
| Total quantity of | 1.3 | 0.99 | 1.05 | 1.04 |
| impurities | | | | |
| formed, | | | | |
| % by HPLC | | | | |

*not according to the invention
^{#}according to the invention

Embodiments and/or optional features of the invention have been described above. Any aspect of the invention may be combined with any other aspect of the invention, unless the context demands otherwise. Any of the embodiments or optional features of any aspect may be combined, singly or in combination, with any aspect of the invention, unless the context demands otherwise.

The invention will now be described further by reference to the following examples, which are intended to illustrate but not limit, the scope of the invention.

### EXAMPLES

The reactions were carried out under a nitrogen atmosphere.

### Abbreviations

Ethyl ester remifentanil ethyl ester impurity
HCl hydrogen chloride
IPA isopropyl alcohol
IPA ester remifentanil isopropyl ester impurity
M molar
MIBK methyl isobutyl ketone
min minute
N net weight
N/D not detected
Remi acid remifentanil acid impurity
Remi base remifentanil base
RemiHCl remifentanil hydrochloride
RemiPPH remipropanamide
RRT relative retention time
Tech Technical

### HPLC Method

### Remifentanil HPLC Method

System: Thermo Dionex UltiMate 3000
Column: Waters X-Terra RP18 150×4.6 mm, 3.5 microns
Mobile Phase: Prepare a solution as follows:
   : A 770 mg of ammonium acetate dissolved in 1000 ml of water and adjusted to pH 9.0 with ammonia (SG 0.88)
: B HPLC grade acetonitrile
Flow rate: 1.5 ml/minute
Temperature: 40° C.
Detector: UV at 220 nm
Injection Volume: 10 microlitres
Run Time: 40 minutes

### Gradient Profile:

| Time (min) | A % v/v | B % v/v |
|---|---|---|
| 0 | 80 | 20 |
| 25 | 40 | 60 |
| 40 | 40 | 60 |
| 45 | 80 | 20 |
| 50 | 80 | 20 |

### Sample Solution (Test Solution (a))

Weigh accurately 100 mg (±10 mg) of sample into a 10 ml volumetric flask. Dissolve in and dilute to volume with methanol.

### Standard Solution (Remifentanil Solution (b))

Dilute 1.0 ml of the sample solution to 100.0 ml with methanol. Further dilute 1.0m1 to 10.0 ml with methanol.

### System Suitability Solution

Weigh accurately 10 mg (±1 mg) each of Remi-N-benzylpropionamide ester oxalate and N-benzylpropionamide oxalate into a 10 ml volumetric flask. Dissolve in and dilute to volume with methanol

Alternatively, weigh accurately 10 mg (±1 mg) of N-benzylpropionamide oxalate into a 10 ml volumetric flask, add 1.0 ml of sample solution and dissolve in and dilute to volume with methanol.

### Method

Make a blank injection of methanol.

Make duplicate injections of the System Suitability Solution.

The resolution between the peaks due to Remi-N-benzylpropionamide ester and N-benzylpropionamide (relative retention time about 1.09) must be not less than 2 when calculated by the PhEur method.

Make triplicate injections of the standard solution. The relative standard deviation of the peak responses must not exceed 10%.

Calculate the signal to noise ratio for the Remi-N-benzylpropionamide ester peak in the first of the injections of the standard solution. The limit of quantitation calculated from the signal/noise ratio by the Ph.Eur. method must be not greater than the reporting threshold of 0.05%

If the System Suitability requirements are not met repair/replace the column or system or repeat the test with new mobile phase.

Make duplicate injections of the sample solution and average the peak responses.

All samples should be analysed as soon as they are prepared to avoid degradation.

This HPLC method meets the acceptance criteria in the European Pharmacopoeia Monograph for Remifentanil Hydrochloride.

### Unspecified Impurities, %

Unspecified impurities (%)=[Impurity peak area in test solution (a)×0.1]/Mean remifentanil peak area in remifentanil solution (b)

### Example 1 (According to the Invention)

### Preparation of Remifentanil Hydrochloride Using HCl Gassed Methanol

1. 50 ml of MIBK solution containing approximately 5.0 g of remifentanil base (Sample A1) was added to a 250 ml flange flask.
2. A stirrer was set to just over 1 on the dial (about 1.2).
3. HCl gassed methanol (3 M) was added dropwise over 20 minutes. 3.6 ml was added in total. The water content of the HCl gassed methanol was analysed by KF and determined to be 0.75% average.
4. Several additions of HCl gassed methanol was required in order to precipitate out remifentanil hydrochloride. On addition of the HCl gassed methanol, the reaction mixture turned hazy before precipitating as a solid. It was hypothesised that remifentanil HCl is more soluble in methanol than isopropyl alcohol.
5. A sample of the reaction mixture liquors was extracted and the pH tested using water wet 1.7-3.8 pH papers. The pH was determined to be 1.7 (blue).
6. The reaction mixture was stirred for 5 minutes.
7. The solid was filtered and washed with 5 ml MIBK. The solid was pulled dry for 1 hour.
8. The solid appeared very dry. N=3.82 g.
9. The solid was analysed by HPLC (Sample A2).
10. The liquors were analysed (0.5 ml in 5 ml of methanol) by HPLC (Sample A3).
11. The liquors contained a few mls of an opaque orange coloured liquid. This liquid was removed and analysed by HPLC (Sample A4).

### MIBK Slurry

1. An MIBK slurry was formed by placing 1.0 g of Sample A2 in a vial and adding MIBK (10 ml, 10 volumes).
2. The solid was filtered and washed with 1 ml MIBK. The solid was pulled dry for 5 minutes.
3. The solid appeared very dry (Sample A5).
4. The pH of the solid was determined by dissolved 0.5 g of Sample A5 in 50 ml ELGA water. The pH was found to be 6.05.

All samples were prepared with HPLC methanol and analysed using the HPLC method described above. The table below shows the impurity profiles of Samples A1-A5.

| | | Area % | | | | |
|---|---|---|---|---|---|---|
| | | | | | A4 | |
| | | | | | RemiHCl | |
| | | | A2 | A3 | Tech Mother | A5 |
| | | A1 | RemiHCl | RemiHCl | liquors | RemiHCl |
| | | Remi Base | Before | Tech Mother | (orange | Tech After |
| Identity | RRT | in MIBK | Slurry | liquors | liquid) | Slurry |
| | 0.21 | N/D | N/D | N/D | 0.06 | N/D |
| Remi Acid | 0.23 | N/D | N/D | 2.12 | 10.65 | N/D |
| | 0.26 | 1.40 | N/D | N/D | 0.11 | N/D |
| | 0.30 | N/D | 0.04 | N/D | 0.04 | 0.01 |
| | 0.39 | N/D | N/D | N/D | 0.16 | N/D |
| MIBK | 0.41 | 1.24 | N/D | 43.20 | 0.31 | 0.02 |
| MIBK | 0.51 | 0.29 | N/D | 7.39 | 0.30 | N/D |
| | 0.69 | 0.08 | 0.08 | N/D | N/D | 0.08 |
| Remifentanil | 1.00 | 96.73 | 99.68 | 36.70 | 85.12 | 99.76 |
| | 1.04 | N/D | N/D | 0.14 | 0.15 | N/D |
| | 1.11 | N/D | N/D | N/D | 0.40 | N/D |
| | 1.22 | N/D | N/D | N/D | 0.67 | N/D |
| | 1.28 | N/D | N/D | N/D | 0.11 | N/D |
| | 1.34 | 0.05 | N/D | 0.61 | N/D | N/D |
| | 1.44 | N/D | N/D | 0.59 | N/D | N/D |
| | 1.52 | N/D | N/D | N/D | 0.12 | N/D |
| | 1.60 | N/D | N/D | N/D | 0.06 | N/D |
| | 1.65 | N/D | N/D | 0.70 | 0.04 | N/D |
| | 1.86 | N/D | N/D | 1.57 | 0.47 | 0.01 |
| | 1.90 | 0.05 | 0.20 | N/D | N/D | N/D |
| | 1.96 | N/D | N/D | 0.32 | N/D | N/D |
| | 1.98 | 0.08 | N/D | N/D | 0.83 | 0.06 |
| | 2.06 | 0.01 | N/D | 0.43 | 0.15 | 0.02 |
| | 2.14 | 0.03 | N/D | N/D | 0.18 | 0.03 |
| | 2.38 | 0.03 | N/D | 4.78 | 0.02 | N/D |
| | 2.42 | 0.01 | N/D | 1.46 | N/D | N/D |

### Example 2 (According to the Invention)

### Experiment With Excess Acid and High Water Content

1. 50 ml of MIBK solution containing approximately 5.0 g of remifentanil base (Sample B1) was added to a 250 ml flange flask.
2. A stirrer was set to just over 1 on the dial (about 1.2).
3. HCl gassed methanol (3 M) was added dropwise over 20 minutes. 3.6 ml was added in total. The water content of the HCl gassed methanol was analysed by KF and determined to be 1.33% average.
4. Several additions of HCl gassed methanol was required in order to precipitate out remifentanil hydrochloride. On addition of the HCl gassed methanol, the reaction mixture turned hazy before precipitating as a solid.
5. A sample of the reaction mixture liquors was extracted and the pH tested using water wet 1.7-3.8 pH papers. The pH was determined to be 1.7 (blue).
6. A further 3 ml HCl gassed methanol was added slowly and stirred for 5 minutes.
7. The solid was filtered and washed with 5 ml MIBK (1 volume). The solid was pulled dry for 1 hour.
8. The solid appeared very dry. N=3.8 g.
9. The solid was analysed by HPLC (Sample B2).
10. The liquors were analysed (0.5 ml in 5 ml of methanol) by HPLC (Sample B3).
11. The liquors contained a few mis of an opaque orange coloured liquid settling on the bottom. This liquid was removed and analysed by HPLC (Sample B4).

### MIBK Slurry

1. An MIBK slurry was formed by placing 2.0 g of Sample B2 in a vial and adding MIBK (20 ml, 10 volumes).
2. The solid was filtered and washed with 2 ml MIBK. The solid was pulled dry for 5 minutes.
3. The solid appeared very dry (Sample B5).
4. The pH of the solid was determined by dissolved 0.5 g of Sample B5 in 50 ml ELGA water. The pH was found to be 6.09.

All samples were prepared with HPLC methanol and analysed using the HPLC method described above. The table below shows the impurity profiles of Samples B1-B5.

| | | Area % | | | | |
|---|---|---|---|---|---|---|
| | | | | | B4 | |
| | | | | | RemiHCl | |
| | | | B2 | B3 | Tech Mother | B5 |
| | | B1 | RemiHCl | RemiHCl | liquors | RemiHCl |
| | | Remi Base | Before | Tech Mother | (orange | Tech After |
| Identity | RRT | in MIBK | Slurry | liquors | liquid) | Slurry |
| | 0.21 | N/D | N/D | 0.71 | N/D | N/D |
| Remi Acid | 0.23 | N/D | N/D | 1.48 | 9.20 | N/D |
| | 0.26 | 1.30 | N/D | 0.46 | 0.04 | N/D |
| | 0.31 | N/D | N/D | N/D | N/D | 0.01 |
| | 0.36 | N/D | N/D | 0.34 | N/D | N/D |
| | 0.38 | | N/D | 0.22 | N/D | N/D |
| MIBK | 0.41 | 1.18 | 0.01 | 55.45 | 0.23 | 0.02 |
| | 0.49 | N/D | N/D | N/D | 0.04 | N/D |
| MIBK | 0.51 | 0.28 | N/D | 11.59 | 0.01 | N/D |
| | 0.69 | 0.05 | 0.09 | N/D | N/D | 0.08 |
| | 0.89 | N/D | N/D | N/D | 0.05 | N/D |
| Remifentanil | 1.00 | 97.19 | 99.90 | 11.83 | 88.03 | 99.88 |
| | 1.11 | N/D | N/D | N/D | 0.24 | N/D |
| | 1.22 | N/D | N/D | N/D | 0.46 | N/D |
| | 1.28 | N/D | N/D | N/D | 0.06 | N/D |
| | 1.30 | N/D | N/D | N/D | 0.04 | N/D |
| | 1.34 | N/D | N/D | 0.37 | N/D | N/D |
| | 1.44 | N/D | N/D | 1.23 | N/D | N/D |
| | 1.54 | N/D | N/D | N/D | 0.14 | N/D |
| | 1.90 | N/D | N/D | N/D | 0.55 | N/D |
| | 2.00 | N/D | N/D | N/D | 0.59 | N/D |
| | 2.05 | N/D | N/D | N/D | 0.10 | N/D |
| | 2.17 | N/D | N/D | N/D | 0.22 | N/D |
| | 2.37 | N/D | N/D | 12.14 | N/D | N/D |
| | 2.41 | N/D | N/D | 4.17 | N/D | N/D |

### Example 3 (According to the Invention)

### Experiment With an Ethanol Spike and High Water Content

1. 50 ml of MIBK solution containing approximately 5.0 g of remifentanil base (Sample C1) was added to a 250 ml flange flask.
2. A stirrer was set to just over 1 on the dial (about 1.2).
3. Ethanol (1 ml, 2% spike) was added.
4. HCl gassed methanol (3M) was added dropwise over 20 minutes. 3.6 ml was added in total. The water content of the HCl gassed methanol was analysed by KF and determined to be 1.6% average.
5. Several additions of HCl gassed methanol was required in order to precipitate out remifentanil hydrochloride. On addition of the HCl gassed methanol, the reaction mixture turned hazy before precipitating as a solid.
6. A sample of the reaction mixture liquors was extracted and the pH tested using water wet 1.7-3.8 pH papers. The pH was determined to be 1.7 (blue).
7. The reaction mixture was stirred for 5 minutes.
8. The solid was filtered and washed with 5 ml MIBK (1 volume). The solid was pulled dry for 1 hour.
9. The solid appeared very dry. N=3.89 g.
10. The solid was analysed by HPLC (Sample C2).
11. The liquors were analysed (0.5 ml in 5 ml of methanol) by HPLC (Sample C3).
12. The liquors contained a few mis of an opaque orange coloured liquid settling on the bottom. This liquid was removed and analysed by HPLC (Sample C4).

### MIBK Slurry

1. An MIBK slurry was formed by placing 2.0 g of Sample C2 in a vial and adding MIBK (20 ml, 10 volumes).
2. The solid was filtered and washed with 2 ml MIBK. The solid was pulled dry for 5 minutes.
3. The solid appeared very dry (Sample C5).
4. The pH of the solid was determined by dissolved 0.5 g of Sample C5 in 50 ml ELGA water. The pH was found to be 5.98.

All samples were prepared with HPLC methanol and analysed using the HPLC method described above. The table below shows the impurity profiles of Samples C1-C5.

| | | Area % | | | | |
|---|---|---|---|---|---|---|
| | | | | | Sample | |
| | | | | | C4 | |
| | | | | Sample | RemiHCl | |
| | | | Sample | C3 | Tech | Sample |
| | | Sample | C2 | RemiHCl | Mother | C5 |
| | | C1 | RemiHCl | Tech | liquors | RemiHCl |
| | | Remi Base | Before | Mother | (orange | Tech After |
| Identity | RRT | in MIBK | Slurry | liquors | liquid) | Slurry |
| | 0.21 | N/D | N/D | N/D | 0.03 | N/D |
| Remi Acid | 0.23 | N/D | N/D | 1.22 | 4.44 | N/D |
| | 0.26 | 1.40 | N/D | 0.40 | 0.03 | N/D |
| | 0.36 | N/D | N/D | 0.21 | 0.03 | N/D |
| | 0.38 | N/D | N/D | 0.34 | 0.03 | N/D |
| MIBK | 0.41 | 1.21 | 0.01 | 49.87 | 0.13 | 0.01 |
| | 0.45 | N/D | N/D | N/D | 0.05 | N/D |
| MIBK | 0.51 | 0.29 | N/D | 7.96 | 0.05 | N/D |
| | 0.69 | 0.04 | 0.04 | N/D | 0.02 | 0.05 |
| | 0.89 | N/D | N/D | N/D | 0.01 | N/D |
| | 0.90 | N/D | N/D | N/D | 0.02 | N/D |
| Remifentanil | 1.00 | 96.99 | 99.95 | 39.35 | 93.19 | 99.92 |
| | 1.13 | N/D | N/D | N/D | 0.12 | N/D |
| | 1.24 | N/D | N/D | N/D | 0.42 | 0.02 |
| | 1.28 | N/D | N/D | N/D | 0.09 | N/D |
| | 1.35 | 0.07 | N/D | 0.66 | 0.01 | N/D |
| | 1.55 | N/D | N/D | N/D | 0.17 | N/D |
| | 1.90 | N/D | N/D | N/D | 0.21 | N/D |
| | 2.02 | N/D | N/D | N/D | 0.73 | N/D |
| | 2.19 | N/D | N/D | N/D | 0.19 | N/D |

Samples of Remifentanil HCl produced in the laboratory using the pre-gassed HCl methanol solution (i.e. Example 1 Sample A5, Example 2 Sample B5, and Example 3 Sample C5) were compared to batches of remifentanil HCl manufactured by directly gassing with hydrogen chloride:

| | | Area % | | | | | |
|---|---|---|---|---|---|---|---|
| | | Examples* | | | | | |
| | | Example 1 | Example 2 | Example 3 | | | |
| | | Sample | Sample | Sample | Direct gassing batches^{#} | | |
| Identity | RRT | A5 | B5 | C5 | 1 | 2 | 3 |
| Remi Acid | 0.23 | N/D | N/D | N/D | 0.06 | 0.03 | 0.02 |
| | 0.30 | N/D | N/D | N/D | 0.11 | 0.07 | 0.04 |
| RemiPPH | 0.42 | N/D | N/D | N/D | 0.05 | 0.05 | 0.04 |
| | 0.53 | N/D | N/D | N/D | 0.04 | 0.03 | 0.05 |
| | 0.68 | 0.08 | 0.08 | 0.05 | N/D | 0.02 | 0.08 |
| Remifentanil | 1.00 | 99.76 | 99.88 | 99.92 | 99.42 | 99.60 | 99.61 |
| Ethyl Ester | 1.26 | N/D | N/D | 0.02 | 0.07 | 0.05 | 0.04 |
| Isopropyl ester | 1.51 | N/D | N/D | N/D | 0.02 | 0.04 | 0.02 |
| | 1.71 | N/D | N/D | N/D | 0.11 | 0.05 | 0.05 |
| | 1.79 | N/D | N/D | N/D | 0.12 | 0.06 | 0.03 |
| Total impurities^{§} | | 0.24 | 0.12 | 0.07 | 0.58 | 0.40 | 0.39 |

*according to the invention
^{#}not according to the invention
^{§}unknown peaks < 0.05 area % omitted from table but the area % of the total impurities include all peaks

The impurity profiles for the remifentanil base input for Examples 1-3 are similar, showing that all reactions had a similar starting profile.

All remifentanil HCl samples isolated i.e. A5, B5, and C5, show clean products were obtained. The total quantity of impurities present in Samples A5, B5, and C5 (0.24%, 0.12% and 0.07% respectively) are significantly lower than in the batches produced by directly gassing with hydrogen chloride gas (0.58%, 0.40% and 0.39% respectively).

Ethanol may be present in MIBK as a residual impurity and may be introduced into the reaction mixture as a consequence of using MIBK. It may then react in the presence of additional HCl to form the ethyl ester impurity. The ethyl ester impurity was not identified in significantly higher levels in Example 3 Sample C5 despite the reaction being spiked with ethanol.

### Example 4 (Not According to the Invention)

### Stability of Remifentanil HCl Technical and Remifentanil HCl Pure in Isopropyl Alcohol

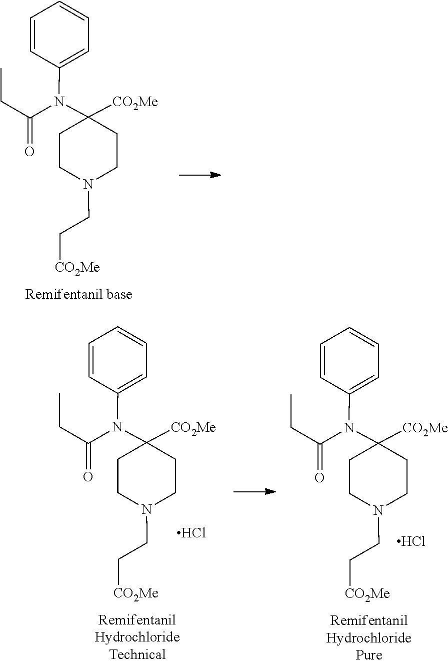 [SMILES: CCC(=O)N(c1ccccc1)C1(C(=O)OC)CCN(CCC(=O)OC)CC1 ; CCC(=O)N(c1ccccc1)C1(C(=O)OC)CCN(CCC(=O)OC)CC1 ; Cl ; CCC(=O)N(c1ccccc1)C1(C(=O)OC)CCN(CCC(=O)OC)CC1 ; Cl]

Remifentanil HCl Technical may be prepared by gassing a solution of remifentanil base in methyl isobutyl ketone (MIBK) with hydrogen chloride gas directly. The reaction mixture may be acidified to a pH range of about 1.5 to about 3.0 by the addition of the HCl gas. Remifentanil HCl Technical precipitates out of solution and is isolated by filtration. Remifentanil HCl may then be further purified by recrystallisation from isopropyl alcohol (IPA) to provide Remifentanil Hydrochloride Pure.

Samples were taken of several batches of Remifentanil HCl Technical and Pure produced in this way. The stability of the samples were assessed as described below:

### Stress Test For Remifentanil HCl Using Recrystallisation Conditions

The following was carried out with a range of Remifentanil HCl Technical and Pure samples:
1. Remifentanil HCl (0.5 g) was charged to a carousel tube.
2. IPA (6 mL) was charged to the tube.
3. A magnetic stirrer was added to the tube.
4. The carousel was switched on and heated to reflux (82° C.).
5. Further IPA was added to some samples and an additional 4 mL IPA was added to all tubes.
6. A solution formed.
7. A sample was taken at Time 0 (approx. 1 mL in 20 mL methanol).
8. A sample was taken after 2 hours had lapsed (approx. 1 mL in 20 mL methanol).
9. The solution was held at temperature overnight.
10. A sample was taken after 24 hours had lapsed (approx. 1 mL in 20 mL methanol).
11. The sample was left to cool and disposed of appropriately.

Samples used:

| | Remifentanil HCl Technical or |
|---|---|
| Sample | Remifentanil HCl Pure |
| A | Technical |
| B | Technical |
| C | Technical |
| D | Technical |
| E | Technical |
| F | Technical |
| G | Technical |
| H | Pure |
| I | Pure |
| J | Pure |

12. Samples of each batch (1.25 g) were dissolved in 25 mL water. Certain samples showed a haze which dissolved on addition of HCl. The samples were analysed by the HPLC method provided above:

| | | | RemiPPH by area % | | |
|---|---|---|---|---|---|
| | | Solution | Time Zero | 2 hours | 24 hours |
| Sample | Stage | Colour | (1) | (2) | (3) |
| A | Technical | Haze | 0.03 | 0.11 | 1.18 |
| B | Technical | Haze | 0.03 | 0.35 | 2.90 |
| C | Technical | Less hazy* | 0.04 | 0.33 | 2.67 |
| D | Technical | Haze* | 0.05 | 0.16 | 0.89 |
| E | Technical | Haze | 0.05 | 0.20 | 1.31 |
| F | Technical | Haze* | 0.04 | 0.23 | 1.18 |
| G | Technical | Sharp | 0.01 | 0.01 | 0.04 |
| H | Pure | Sharp | 0.07 | 0.07 | 0.19 |
| I | Pure | Sharp | 0.11 | 0.16 | 0.55 |
| J | Pure | Sharp | 0.09 | 0.12 | 0.30 |
| K | Recrystallised | Sharp | 0.03 | 0.05 | 0.09 |
| | Pure | | | | |

*Haze removed on addition of HCl (aqueous)

HPLC related substances data after 24-hour stress test:

| | | Area % | | | | | | | | | | |
|---|---|---|---|---|---|---|---|---|---|---|---|---|
| | RRT | A | B | C | D | E | F | G | H | I | J | K |
| | 0.20 | 0.02 | N/D | N/D | N/D | N/D | N/D | 0.11 | N/D | N/D | N/D | N/D |
| Remi Acid | 0.23 | 0.15 | 0.10 | 0.04 | 0.11 | 0.07 | 0.03 | 0.68 | 0.02 | N/D | 0.02 | 0.06 |
| | 0.26 | 0.12 | 0.27 | 0.24 | 0.11 | 0.16 | 0.14 | N/D | N/D | 0.03 | 0.04 | N/D |
| | 0.30 | 0.04 | 0.04 | 0.04 | 0.09 | 0.09 | 0.07 | 0.08 | 0.05 | 0.04 | 0.04 | 0.04 |
| | 0.37 | 0.01 | 0.01 | N/D | N/D | N/D | N/D | N/D | N/D | N/D | N/D | N/D |
| RemiPPH | 0.42 | 1.18 | 2.90 | 2.67 | 0.89 | 1.31 | 1.18 | 0.04 | 0.19 | 0.55 | 0.30 | 0.09 |
| | 0.51 | 0.04 | 0.04 | 0.70 | N/D | 0.05 | 0.03 | N/D | N/D | N/D | N/D | N/D |
| | 0.68 | N/D | N/D | N/D | N/D | N/D | 0.08 | 0.08 | N/D | N/D | 0.06 | N/D |
| | 0.77 | 0.02 | N/D | N/D | N/D | N/D | N/D | N/D | N/D | N/D | N/D | 0.05 |
| Remifentanil | 1.00 | 97.30 | 95.94 | 96.16 | 97.92 | 97.53 | 98.02 | 87.99 | 99.15 | 99.04 | 99.27 | 99.20 |
| | 1.05 | 0.06 | N/D | N/D | N/D | N/D | N/D | N/D | N/D | N/D | N/D | N/D |
| | 1.26 | 0.12 | 0.08 | 0.08 | 0.10 | 0.07 | 0.05 | 0.13 | N/D | N/D | N/D | N/D |
| Isopropyl ester | 1.51 | 0.25 | 0.41 | 0.40 | 0.27 | 0.29 | 0.23 | 9.75 | 0.20 | 0.19 | 0.12 | 0.34 |
| | 1.53 | 0.15 | N/D | 0.06 | 0.15 | 0.13 | N/D | 0.13 | 0.10 | N/D | N/D | 0.03 |
| | 1.60 | 0.06 | N/D | N/D | N/D | 0.04 | N/D | 0.05 | 0.03 | N/D | 0.02 | 0.03 |
| | 1.71 | 0.19 | 0.11 | 0.10 | 0.23 | 0.16 | 0.12 | 0.18 | 0.17 | 0.10 | 0.08 | 0.09 |
| | 1.74 | 0.10 | 0.05 | 0.04 | 0.09 | 0.08 | 0.05 | 0.07 | 0.08 | 0.04 | 0.06 | 0.03 |
| | 1.78 | 0.12 | 0.03 | 0.02 | 0.04 | 0.02 | N/D | N/D | N/D | N/D | N/D | N/D |
| Total | | 2.63 | 4.04 | 4.39 | 2.08 | 2.47 | 1.98 | 11.3 | 0.84 | 0.95 | 0.74 | 0.76 |
| impurities | | | | | | | | | | | | |

The remipropanamide (RemiPPH) levels increased in all batches except for samples taken from batches G and H. Without wishing to be bound by theory, it is believed that if the cause of the formation of remipropanamide is the presence of remifentanil base, then batches G and H contain no remifentanil base. It was confirmed that these batches had the greatest volume of HCl gas added to them during the salt formation.

When HCl (aqueous) acid was added to samples C, D and F, the haze was removed which indicates the presence of remifentanil base. The pH of solution E and H were checked using pH papers. The pH of solution E was 5, whilst that of solution H was 4. This further confirms the theory that the Technical samples contain remifentanil base.

The formation of remipropanamide is faster in the Technical samples than the Pure samples indicating a higher percentage of remifentanil base in the technical samples. Without wishing to be bound be theory, it is believed that the recrystallisation procedure removes remifentanil base, however, the high temperatures used during the recrystallisation procedure may promote remipropanamide formation.

### Example 5 (According to the Invention)

### Undergassing With HCl Gassed Methanol

1. 50 mL of MIBK solution was added to a 250 mL flange flask. The solution contained approx. 5.0 g of remifentanil base (Sample A1).
2. The stirrer was set to just over 1 on the dial (approx. 1.2).
3. HCl gassed methanol (2M) was added dropwise and 3 mL was added in total. The methanol had a water content of 0.22%. It was prepared 5 weeks prior to the experiment and had been stored in the freezer to prevent water generation.
4. A sample of the reaction mixture liquors was extracted and the pH tested with water wet pH 1-14 papers. The pH was 5. The pH for remifentanil HCl formation is typically in the range of 1.5-3. A pH of 5 is higher than the typical range and would be expected to be so if the reaction is undergassed.
5. The reaction mixture was stirred for 5 mins.
6. The resulting solid was filtered and washed with 5 mL MIBK. The solid was pulled dry over 1 hour.
7. The solid appeared very dry. N=3.98 g.
8. The solid was analysed by HPLC (Sample A2).
9. The liquors were sampled and analysed by HPLC (0.5 mL in 10 mL ethanol) (Sample A3).

### MIBK Slurry

1. The solid was not removed from the flask. MIBK was added directly to the flask and slurried in situ. MIBK (40 mL, 10 volumes) added.
2. The solid was filtered and pulled dry for 5 minutes.
3. The solid appeared very dry (Sample A4). N=3.93 g.
4. The pH was determined by dissolving 0.5 g of Sample A4 in 50 mL ELGA water. The pH was 5.91.

All samples were prepared with HPLC methanol and analysed using the HPLC method described above.

### Overgassing With HCl Gassed Methanol

1. 50 mL of MIBK solution was added to a 250 mL flange flask. The solution contained approx. 5.0 g of remifentanil base (Sample B1). Not sampled for HPLC.
2. The stirrer was set to just over 1 on the dial (approx. 1.2).
3. HCl gassed methanol (2M) was added dropwise and 7 mL was added in total. The methanol had a water content of 0.22%. It was prepared 5 weeks prior to the experiment and had been stored in the freezer to prevent water generation.
4. A sample of the reaction mixture liquors was extracted and the pH tested with water wet pH 1-14 papers. The pH was 0. The pH for remifentanil HCl formation is typically in the range of 1.5-3. A pH of 0 is lower than the typical range and would be expected to be so if the reaction is overgassed.
5. The reaction mixture was stirred for 5 mins.
6. The resulting solid was filtered and washed with 5 mL MIBK. The solid was pulled dry over 1 hour.
7. The solid appeared very dry. N=3.23 g.
8. The solid was analysed by HPLC (Sample B2).
9. The liquors were sampled and analysed by HPLC (0.5 mL in 10 mL ethanol) (Sample B3).

### MIBK Slurry

1. The solid was not removed from the flask. MIBK was added directly to the flask and slurried in situ. MIBK (40 mL, 10 volumes) added.
2. The solid was filtered and pulled dry for 5 minutes.
3. The solid appeared very dry (Sample B4). N=2.81 g.
4. The pH was determined by dissolving 0.5 g of Sample B4 in 50 mL ELGA water. The pH was 6.20.

All samples were prepared with HPLC methanol and analysed using the HPLC method described above.

### Stress Test Using Recrystallisation Conditions

1. Remifentanil HCl Technical (Sample A4) was weighed into two carousel tubes.
2. Remifentanil HCl Technical (Sample B4) was weighed into two different carousel tubes.
3. IPA (12.6 mL, 14 volumes) was added to each tube and heated to reflux.
4. Once at reflux, a tube containing Sample A4 and a tube containing Sample B4 were sampled (Samples A5 and B5 respectively), removed from the heat and allowed to cool to room temperature.
5. The solid was isolated, and washed with IPA (1 mL, 1 volume per sample).
6. The mother liquors (Samples A6 and B6 respectively) and the solids (Samples A7 and B7 respectively) were sampled.
7. The remaining two tubes were left to reflux overnight before being sampled and isolated using the same sample codes (A8/B8—at reflux sample; A9/B9—mother liquors; A10/B10 isolated pure).

The sample codes are detailed below. All samples were made using approx. 100 mg remifentanil or 0.5 mL of liquors in 10 mL HPLC methanol. The samples were analysed using the HPLC method detailed above.

| Sample | Description |
|---|---|
| A1 | Remifentanil base in MIBK |
| A2 and B2 | Remifentanil HCl Technical prior to slurry |
| A3 and B3 | Technical mother liquors |
| A4 and B4 | Remifentanil HCl Technical after slurry |
| A5 and B5 | At IPA reflux, prior to isolation after 10 minutes |
| A6 and B6 | Remifentanil HCl Pure mother liquors |
| A7 and B7 | Isolated Remifentanil HCl Pure |
| A8 and B8 | At IPA reflux, prior to isolation after 24 hours |
| A9 and B9 | Remifentanil HCl Pure mother liquors |
| A10 and B10 | Isolated Remifentanil HCl Pure |

HPLC data for isolated solid and reslurry samples—Samples A1, A2, A3, and A4:

| | | Area % | | | |
|---|---|---|---|---|---|
| | | A1 | A2 | A3 | A4 |
| | | Remi | RemiHCl | RemiHCl | RemiHCl |
| | | Base in | Before | Tech Mother | Tech After |
| Identity | RRT | MIBK | Slurry | liquors | Slurry |
| | 0.20 | N/D | N/D | 0.25 | N/D |
| Remi Acid | 0.23 | N/D | N/D | 0.21 | N/D |
| MIBK | 0.26 | 2.26 | N/D | 13.52 | N/D |
| | 0.30 | 0.06 | N/D | 0.37 | N/D |
| | 0.38 | N/D | N/D | 0.62 | N/D |
| MIBK | 0.41 | 0.93 | N/D | 8.99 | N/D |
| | 0.45 | 0.08 | N/D | 0.05 | N/D |
| | 0.49 | N/D | N/D | 0.17 | N/D |
| MIBK | 0.51 | 0.23 | N/D | 2.71 | N/D |
| | 0.69 | 0.06 | 0.07 | N/D | 0.07 |
| Remifentanil | 1.00 | 95.86 | 99.69 | 70.67 | 99.89 |
| | 1.26 | N/D | 0.04 | N/D | N/D |
| | 1.36 | 0.24 | N/D | 1.52 | N/D |
| | 1.50 | 0.05 | 0.08 | N/D | N/D |
| | 1.86 | 0.19 | N/D | 0.94 | N/D |

Without wishing to be bound by theory, it is believed that stabilisers in the MIBK are detectable using the described HPLC method. As a result, MIBK is shown as having three RRTs in the HPLC data.

HPLC data for isolated solid and reslurry samples—samples B2, B3, and B4:

Area %

| | | B2 | B3 | B4 |
|---|---|---|---|---|
| | | RemiHCl | RemiHCl | RemiHCl |
| | | Before | Tech Mother | Tech After |
| Identity | RRT | Slurry | liquors | Slurry |
| | 0.17 | N/D | 0.13 | N/D |
| Remi Acid | 0.23 | N/D | 0.13 | N/D |
| MIBK | 0.26 | N/D | 20.86 | N/D |
| | 0.30 | 0.08 | 0.57 | N/D |
| MIBK | 0.41 | N/D | 8.32 | N/D |
| | 0.49 | N/D | 0.18 | N/D |
| MIBK | 0.51 | N/D | 2.35 | N/D |
| | 0.68 | 0.07 | N/D | 0.07 |
| Remifentanil | 1.00 | 99.86 | 64.62 | 99.71 |
| | 1.25 | N/D | 0.18 | 0.04 |
| | 1.36 | N/D | 2.27 | N/D |
| | 1.50 | N/D | N/D | 0.06 |
| | 1.67 | N/D | 0.39 | 0.06 |

HPLC data for samples taken during the stress tests—samples A5-A10, and B5-B10:

| | | Area % | | | | | |
|---|---|---|---|---|---|---|---|
| | | under gassed, 10 minutes reflux | | | | | |
| | | A5 | | | under gassed, 24 hours reflux | | |
| | | Slurry | A6 | | A8 | A9 | |
| | | after 10 | RemiHCl | | Slurry | RemiHCl | |
| | | mins | Pure | A7 | after 24 | Pure | A10 |
| | | IPA | Mother | RemiHCl | hours IPA | Mother | RemiHCl |
| Identity | RRT | Reflux | liquors | Pure | Reflux | liquors | Pure |
| Remi acid | 0.25 | N/D | N/D | N/D | 0.07 | 0.16 | N/D |
| | 0.30 | N/D | N/D | 0.10 | N/D | 0.11 | N/D |
| RemiPPH | 0.45 | N/D | N/D | N/D | 0.60 | 1.32 | 0.33 |
| | 0.68 | 0.06 | 0.05 | 0.13 | 0.07 | 0.10 | 0.05 |
| | 0.71 | N/D | N/D | 0.07 | N/D | N/D | N/D |
| Remifentanil | 1.00 | 99.68 | 99.81 | 99.40 | 98.88 | 97.62 | 99.46 |
| | 1.25 | N/D | N/D | N/D | 0.04 | N/D | N/D |
| IPA ester | 1.50 | 0.10 | N/D | N/D | 0.21 | 0.32 | 0.09 |
| | 1.67 | 0.12 | N/D | N/D | 0.05 | 0.26 | N/D |
| | 1.70 | N/D | N/D | N/D | N/D | 0.11 | N/D |
| | 1.75 | N/D | N/D | N/D | N/D | N/D | N/D |
| | | Area % | | | | | |
| | | over gassed, 10 minutes reflux | | | | | |
| | | B5 | | | over gassed, 24 hours reflux | | |
| | | Slurry | B6 | | B8 | B9 | |
| | | after 10 | RemiHCl | | Slurry | RemiHCl | |
| | | mins | Pure | B7 | after 24 | Pure | B10 |
| | | IPA | Mother | RemiHCl | hours IPA | Mother | RemiHCl |
| Identity | RRT | Reflux | liquors | Pure | Reflux | liquors | Pure |
| Remi acid | 0.25 | N/D | N/D | N/D | 0.06 | 0.13 | N/D |
| | 0.30 | 0.06 | 0.11 | N/D | N/D | 0.11 | N/D |
| RemiPPH | 0.45 | N/D | 0.09 | N/D | 0.48 | 1.05 | 0.25 |
| | 0.68 | 0.08 | 0.09 | 0.05 | 0.06 | 0.10 | 0.05 |
| | 0.71 | N/D | N/D | N/D | N/D | N/D | N/D |
| Remifentanil | 1.00 | 99.56 | 99.38 | 99.73 | 99.17 | 98.02 | 99.58 |
| | 1.25 | N/D | N/D | N/D | N/D | N/D | N/D |
| IPA ester | 1.50 | N/D | 0.07 | 0.05 | 0.12 | 0.28 | 0.10 |
| | 1.67 | 0.20 | 0.18 | N/D | N/D | N/D | N/D |
| | 1.70 | N/D | 0.08 | N/D | N/D | N/D | N/D |
| | 1.75 | 0.09 | N/D | N/D | N/D | N/D | N/D |

### Conclusions:

### Formation of Remifentanil HCl Technical:

Remipropanamide is only detected in the “undergassed” process and is entirely lost to the liquors. The IPA ester is detected at higher levels in the overgassed process, which agrees with previous findings that additional HCl catalyses the reaction between remifentanil base and IPA.
The pH values of the obtained remifentanil HCl Technical samples are similar and as expected. This indicates that excess acid was not trapped in the crystals.

### Recrystallisation: Formation of Remifentanil HCl Pure

When the undergassed sample (Sample A4) was heated in IPA and isolated after 10 minutes, no remipropanamide was detected. When this hold time was increased to 24 hours, the in situ level of remipropanamide increased from 0.60% leaving 0.33% present in the solid.
When repeated with the overgassed material (Sample B4), the remipropanamide level is again undetected in the isolated solid (although the IPA ester was present). When the reflux time was increased to 24 hours, the remipropanamide level increased to 0.48% leaving 0.25% in the solid. The IPA ester is detected in all samples when using the overgassed material.

A comparison can be made between the refluxing sample taken after 24 hours (Samples A8/B8) and samples taken in Example 4. In Example 4, samples of various Remifentanil HCl Technical and Pure were placed in IPA and refluxed for 24 hours. The remipropanamide levels were between 1 and 3%, far higher than the 0.60% and 0.48% observed in this Example. This indicates that the method of the invention results in less unreacted remifentanil base remaining in the reaction mixture, even when insufficient hydrogen chloride (Sample A8) and excess hydrogen chloride (Sample B8) has been used. Comparing the levels of the IPA ester impurity (0.23%-9.75% in Example 4) with the 0.21% and 0.12% shows the pre-gassed methanol route is also favourable for preventing the formation of this impurity.

This Example shows that to reduce the remipropanamide level, sufficient HCl must be added, and that it is desirable for the recrystallisation to be as quick as possible. Comparing with Example 4, the method of the invention results in less remipropanamide being formed.

Without wishing to be bound by theory, it is believed that residual methanol introduced in the remifentanil hydrochloride formation step may give the product more stability in the IPA recrystallisation step. In this regard, residual methanol will be present even if the remifentanil HCl is dried before recrystallisation.

### Example 6 (According to the Invention)

### Rate of Addition of the Pregassed HCl/Methanol Solution to the Remifentanil Base

### Ammonium Phosphate Buffer Preparation

A 2M solution of phosphoric acid was prepared by diluting 68.5 mL of phosphoric acid BP to 500 mL with water. The pH was then adjusted to pH 6.8 with ammonia solution (S.G. 0.88).

### Procedure For the Preparation of Remifentanil Base

1. Remifentanil hydrochloride (49.4 g) was charged to a 1 L flange flask fitted with an overhead stirrer and temperature probe.
2. Water (ELGA, 500 mL) and MIBK (813 mL) were added and the mixture stirred.
3. The pH of the slurry was adjusted using ammonium hydroxide (S.G. 0.88). Target pH was 8.5 to 9.0 but this was exceeded. The pH was found to be 9.5 to 10.0 (24 mL added). The layers were hazing slightly and so the flask was heated to 25° C. and a solution was achieved and the separation was clear. Extra ammonia may have been needed as this batch of remifentanil HCl was found to be more acidic than other batches.
4. The mixture was transferred to a separating funnel. The lower aqueous layer was separated and the MIBK layer transferred to a separate flask.
5. The aqueous layer was returned to the separating funnel and extracted with MIBK (100 mL).
6. The MIBK layers were combined.
7. The combined MIBK layers were extracted with pH 6.8 ammonium phosphate buffer (2×100 mL).
8. The combined MIBK layers were extracted with water (100 mL).
9. Sodium sulfate was added to dry off any water.
10. The solution was filtered to remove the sodium sulfate.
11. The sodium sulfate was washed with MIBK (10 mL). This wash solvent was then added to the combined MIBK layers.

A second solution of remifentanil base in MIBK was made using substantially the same procedure described above except that the starting mass of remifentanil hydrochloride was 100.0 g. The first and second solutions of remifentanil base in MIBK were combined.

### Preparation of 3M HCl in Methanol

1. Methanol (200 mL) was measured into a Schott bottle (gross weight with methanol=393.9 g).
2. The charged bottle was cooled to 5° C.
3. With Parafilm™ over the top of the flask, HCl gas was bubbled through the methanol.
4. This was continued until the weight of the methanol had increased by 53 g.
5. 290 mL methanol was added to the flask to adjust the concentration of HCl down to 3M (3 molar solution was calculated to be 109.38 g per litre (i.e. 21.87 g per 200 mL)).
6. The charged bottle was stored in the fridge and vial containing approx. 20 mL were extracted and used when required.

Karl Fisher analysis was carried out before use and is detailed in the experiment.

Remifentanil hydrochloride formation—Bulk Addition (Not According to the Invention)
1. 300 mL of the remifentanil base MIBK solution was added to a 1 L flange flask. The solution contained approx. 30 g of remifentanil base. Some material had precipitated out on standing and the bottle was placed in a 65° C. oven for 5 minutes to re-dissolve the solid. A sample of the solution was taken (Sample A1).
2. The stirrer was set to just over 1 on the dial (approx. 1.2).
3. HCl gassed methanol (3M) was added in one portion (30 mL). The water content was determined to be KF=0.09%). A haze was observed which resulted in solid precipitating out of solution. The temperature was increased from 25° C. to 32° C. and the flask filled with gas. The pH was tested using pH 1.7-3.8 papers and the pH was determined to be 1.7 (blue). The pH using a pH meter was 2.54.
4. The reaction mixture was stirred for 5 mins.
5. The obtained solid was filtered and washed with 30 mL MIBK (1 volume). The solid was pulled dry. A sample was removed. HPLC and pH analysis of the solid was carried out (Sample A2). The HPLC analysis is below. The pH was 4.22.
6. The solid was washed a second time with 30 mL MIBK (1 volume). The solid was pulled dry over 5 mins. A sample was removed. HPLC and pH analysis of the solid was carried out (Sample A3). The HPLC analysis is below. The pH was 4.31.
7. The solid was washed a third time with 30 mL MIBK (1 volume). The solid was pulled dry over 5 mins. A sample was removed. HPLC and pH analysis of the solid was carried out (Sample A4). The HPLC analysis is below. The pH was 4.21.

The displacement washes were carried out by removing vacuum and allowing percolation before reapplying the vacuum.

HPLC analysis was carried out by dissolving 100 mg in 10 mL methanol and the pH was determined by dissolving 0.5 g in 50 mL ELGA water and measuring with a pH meter.

### Remifentanil Hydrochloride Formation—Controlled Addition of Pregassed HCl/Methanol Solution (According to the Invention)

1. 300 mL of the remifentanil base MIBK solution was added to a 1 L flange flask. The solution contained approx. 30 g of remifentanil base. Some material had precipitated out on standing and the bottle was placed in a 65° C. oven for 5 minutes to re-dissolve the solid. A sample of the solution was taken (Sample B1).
2. The stirrer was set to just over 1 on the dial (approx. 1.2).
3. 30 mL of HCl gassed methanol (3M) was added dropwise at a rate of 3 mL/minute over a period of 10 minutes. The water content of the HCl gassed methanol was determined to be KF=0.09%. A haze was observed which resulted in solid precipitating out of solution. The pH of the solution was tested using pH 1.7-3.8 papers and the pH was determined to be 1.7 (blue). The pH using a pH meter was 1.81.
4. The reaction mixture was stirred for 5 mins.
5. The obtained solid was filtered and washed with 30 mL MIBK (1 volume). The solid was pulled dry for approx. 1 hour. A 1.5 g sample was removed. HPLC and pH analysis of the solid was carried out (Sample B2). The HPLC analysis is below. The pH was 5.55.
6. The solid was washed a second time with 30 mL MIBK (1 volume). The solid was pulled dry over 5 mins. A 1.5 g sample was removed. HPLC and pH analysis of the solid was carried out (Sample B3). The HPLC analysis is below. The pH was 5.51.
7. The solid was washed a third time with 30 mL MIBK (1 volume). The solid was pulled dry over 5 mins. A 1.5 g sample was removed. HPLC and pH analysis of the solid was carried out (Sample B4). The HPLC analysis is below. The pH was 5.62.

The displacement washes were carried out by removing vacuum and allowing percolation before reapplying the vacuum.

HPLC analysis was carried out by dissolving 100 mg in 10 mL methanol and the pH was determined by dissolving 0.5 g in 50 mL ELGA water and measuring with a pH meter.

### Stress Test For Remifentanil Hydrochloride Using Recrystallisation Conditions

1. Samples A4, B2, B3, and B4 were charged to carousel tubes (0.5 g each).
2. IPA (10 ml per tube) was added and the tubes heated at reflux for 24 hours.
3. The samples were analysed by diluting 1 mL of the IPA mixture in 10 mL methanol (Samples A4-1, B2-1, B3-1, and B4-1).

HPLC data for samples taken in the bulk addition experiment—Samples A1-A4:

| | | Dump charge addition, | | | |
|---|---|---|---|---|---|
| | | 3 × displacement washes (area %) | | | |
| | | | A2 | A3 | A4 |
| | | A1 | After | After 2nd | After 3rd |
| | | Remi | displacement | displacement | displacement |
| | RRT | base | wash | wash | wash |
| Remi acid | 0.23 | 0.03 | N/D | N/D | N/D |
| MIBK | 0.24 | 0.08 | 0.07 | 0.08 | 0.08 |
| | 0.26 | 0.07 | N/D | N/D | N/D |
| | 0.31 | 0.04 | 0.03 | 0.06 | 0.07 |
| | 0.38 | 0.04 | N/D | N/D | N/D |
| MIBK | 0.41 | 0.85 | 0.01 | 0.01 | 0.01 |
| RemiPPH | 0.43 | 0.67 | N/D | N/D | N/D |
| | 0.49 | 0.06 | N/D | N/D | N/D |
| MIBK | 0.50 | 0.29 | N/D | N/D | N/D |
| | 0.53 | N/D | 0.03 | N/D | N/D |
| | 0.69 | 0.04 | 0.04 | 0.04 | 0.04 |
| Remifentanil | 1.00 | 96.71 | 99.13 | 99.10 | 99.09 |
| Ethyl ester | 1.27 | 0.12 | 0.07 | 0.08 | 0.08 |
| | 1.45 | 0.10 | 0.07 | 0.07 | 0.07 |
| IPA ester | 1.52 | 0.88 | 0.54 | 0.56 | 0.56 |
| | pH | N/A | 4.22 | 4.31 | 4.21 |

HPLC data for samples taken in the controlled addition experiment—Samples B2-B4:

| | | 3 ml/min addition, 3 × displacement wash | | |
|---|---|---|---|---|
| | | (area %) | | |
| | | B2 | B3 | B4 |
| | | After | After 2nd | After 3rd |
| | | displacement | displacement | displacement |
| | RRT | wash | wash | wash |
| Remi acid | 0.24 | 0.08 | 0.07 | 0.08 |
| | 0.31 | 0.02 | 0.02 | 0.01 |
| RemiPPH | 0.43 | 0.02 | 0.02 | 0.02 |
| | 0.53 | 0.02 | 0.02 | 0.02 |
| | 0.69 | 0.05 | 0.04 | 0.05 |
| Remifentanil | 1.00 | 99.12 | 99.13 | 99.10 |
| Ethyl ester | 1.27 | 0.07 | 0.07 | 0.07 |
| | 1.45 | 0.08 | 0.08 | 0.09 |
| IPA ester | 1.52 | 0.55 | 0.55 | 0.56 |
| | pH | 5.55 | 5.51 | 5.62 |

HPLC data for samples taken after the stress tests—Samples A4-1, B2-1, B3-1, and B4-1:

| | | Extended IPA Heating (24 hours) (area %) | | | |
|---|---|---|---|---|---|
| | | Dump Charge | 3 ml/min | | |
| | | A4-1 | B2-1 | B3-1 | B4-1 |
| | | Final Isolation | After 1st | After 2nd | After 3rd |
| | RRT | (3 washes) | wash | wash | wash |
| Remi acid | 0.23 | 0.13 | 0.08 | 0.09 | 0.09 |
| | 0.25 | N/D | 0.04 | 0.04 | 0.04 |
| | 0.33 | 0.02 | 0.01 | 0.01 | 0.01 |
| RemiPPH | 0.40 | 0.01 | 0.02 | 0.02 | 0.02 |
| | 0.68 | 0.04 | 0.05 | 0.05 | 0.05 |
| Remifentanil | 1.00 | 98.69 | 98.68 | 98.55 | 98.6 |
| Ethyl Ester | 1.25 | 0.07 | 0.07 | 0.07 | 0.07 |
| | 1.43 | 0.09 | 0.07 | 0.08 | 0.08 |
| IPA ester | 1.51 | 0.94 | 0.65 | 0.69 | 0.68 |
| Total | | 1.3 | 0.99 | 1.05 | 1.04 |
| impurities | | | | | |

### Conclusion

The pH of the solids obtained by adding the HCl/methanol solution in one charge are approx. 1 pH unit lower than those obtained when the HCl/methanol solution is added in a controlled manner. Without wishing to be bound by theory, this suggests that HCl is trapped in the crystals formed. This was confirmed by the HPLC results obtained after the IPA recrystallisation stress tests. When Sample A4 was heated in IPA, the IPA ester level increased from 0.56% to 0.94%, whilst the level in Sample B4 only increased to 0.68% (from 0.56%). Again, without wishing to be bound by theory, it is hypothesised that this is due to excess HCl catalysing the formation of the IPA ester.

Little remipropanamide was detected showing that all remifentanil base was converted to remifentanil HCl. This is an improvement on gassing HCl through a solution of remifentanil base.

This experiment provides further evidence to show the method of the invention gives a more robust process. It also shows that the controlled addition of the present invention is required to prevent the formation of the IPA ester impurity. It is believed that fast addition will form the crystals quickly trapping the HCl, which on recrystallisation will catalyse the formation of the IPA ester.

### Example 7 (According to the Invention)

### Assessing the Stability of Pre-Gassed HCl in Methanol

### Preparation of 3 M HCl in Methanol

1. Methanol (100 g/128 ml) was measured into a Schott bottle (gross weight with methanol was 346.6 g)
2. The charged bottle was cooled using an ice bath.
3. With Parafilm™ over the top of the flask, HCl gas was bubbled through the methanol.
4. This was continued until the methanol weight had increased by at least 16 g.
5. The final increase in weight was 18 g.
6. The bottle was stored in the freezer (temperature ˜18° C.) and approx. 20 ml samples were taken for analysis when required.

### Karl Fisher Analysis

Time zero: 0.088%
1 month: 0.0965%
2 months: 0.122%
3 months: 0.130%

This experiment has shown that although the water content of the HCl pre-gassed methanol increases, even when stored in the freezer, it remains at 0.13% after 3 months. Examples 1-3 demonstrate that high quality remifentanil hydrochloride can be obtained after using HCl gassed methanol which has a water content of 0.75%, 1.33% and 1.6% respectively.

1. A process for the preparation of remifentanil hydrochloride, the process comprising the step of combining a solution of remifentanil base in a ketone solvent with a solution of an alcohol solvent comprising hydrogen chloride, wherein one solution is added at a controlled rate to the other solution to form a reaction mixture comprising a precipitate of remifentanil hydrochloride.

2. The process according to claim 1, wherein the ketone solvent has a boiling point at atmospheric pressure (i.e. 1.0135×10⁵ Pa) below about 160° C., such as below about 120° C.

3. The process according to claim 2, wherein the ketone solvent is selected from the group consisting of methyl ethyl ketone, methyl isobutyl ketone and diethylketone.

4. The process according to claim 3, wherein the alcohol solvent has a boiling point at atmospheric pressure below about 120° C., such as below about 100° C.

5. The process according to claim 4, wherein the alcohol solvent is selected from the group consisting of methanol, ethanol, n-propanol, isopropanol, butanol (n-, i- or t-), pentanols, cyclopentanol, hexanols, cyclohexanol and combinations thereof.

6. The process according to claim 5, wherein the alcohol solvent is methanol.

7. The process according to claim 1, wherein the alcohol solvent comprising hydrogen chloride is prepared by dissolving hydrogen chloride in the alcohol solvent.

8. The process according to claim 1, wherein the hydrogen chloride is present in the reaction mixture in stoichiometric or in slight excess to the remifentanil base.

9. The process according to claim 1, wherein the solution of remifentanil base in a ketone solvent is added at a controlled rate to the solution of an alcohol solvent comprising hydrogen chloride.

10. The process according to claim 1, wherein the solution of an alcohol solvent comprising hydrogen chloride is added at a controlled rate to the solution of remifentanil base in a ketone solvent.

11. The process according to claim 1, wherein the controlled addition is at a substantially constant rate.

12. The process according to claim 1, wherein the temperature of the reaction mixture is maintained at ambient temperature or less during the addition of one solution to the other.

13. The process according to claim 1, further comprising the step of: treating the precipitate of remifentanil hydrochloride with a ketone solvent.

14. The process according to claim 1, further comprising the step of: recrystallizing the precipitate of remifentanil hydrochloride from one or more alcohol solvents.
